(19) United States
(12) Patent Application Publication
(10) Pub. No.: US 2024/0408380 A1
(43) Pub. Date: Dec. 12, 2024

(54) DEVICE AND METHOD FOR THE ELECTRICAL STIMULATION OF BODY REGIONS

(71) Applicant: eaglefit GmbH, Ulm/Langenau (DE)

(72) Inventor: Jochen BORNER, Ulm/Langenau (DE)

(73) Assignee: eaglefit GmbH, Ulm/Langenau (DE)

(21) Appl. No.: 18/807,870

(22) Filed: Aug. 16, 2024

Related U.S. Application Data
(63) Continuation of application No. PCT/EP2023/053649, filed on Feb. 14, 2023.

(30) Foreign Application Priority Data
Feb. 16, 2022 (DE) 10 2022 103 581.6
Feb. 16, 2022 (DE) 20 2022 100 862.0
Mar. 25, 2022 (DE) 10 2022 107 069.7

(51) Int. Cl. A61N 1/04

(52) U.S. Cl. CPC A61N 1/0452; A61N 1/0484

(57) ABSTRACT

A device for stimulating body regions of a user, in particular for electrical muscle stimulation training. The device comprises an article of clothing made of an elastic textile structure with integrated electrodes and integrated electrical supply lines leading to the electrodes, as well as a local control unit for power supply and control. For trouble-free operation of the device, it must be ensured that the control unit is securely connected to the electrodes of the article of clothing during training. For this purpose, the article of clothing has a closable receiver, in particular a pocket, for receiving the control unit. A contacting unit, via which the control unit can be connected to the electrodes, is provided in the interior of the receiver.

This nonprovisional application is a continuation of International Application No. PCT/EP2023/053649, which was filed on Feb. 14, 2023, and which claims priority to German Patent Application No. 10 2022 103 581.6, which was filed in Germany on Feb. 16, 2022 and German Patent Application No. 20 2022 100 862.0, which was filed in Germany on Feb. 16, 2022, and German Patent Application No. 10 2022 107 069.7, which was filed in Germany on Mar. 25, 2022, and which are herein incorporated by reference.

### BACKGROUND OF THE INVENTION

### Field of the Invention

The invention relates to a device for stimulating body regions of a user. The invention further relates to a method for stimulating body regions.

### Description of the Background Art

Stimulation devices are used, for example, for electrical muscle stimulation (EMS) training of selected body regions of a user within the scope of rehabilitation measures or for athletic training. The device comprises an article of clothing which, at least in sections, is made of an elastic textile structure and worn on the body of the person doing training. Situated in the article of clothing is at least one pair of electrodes that bring about the desired electrical muscle stimulation, these electrodes being connected via electrical lines to a voltage source or current source by means of which the electrodes are supplied with electrical power. In addition, the device includes at least one control unit, with the aid of which the voltage supply to the electrodes, with settable pulsations, for example, is controlled.

An article of clothing made of a textile material is known from DE 20 2020 106 704 U1, which is herein incorporated by reference, into which at least one pair of electrodes is integrated. During use of the article of clothing, the electrodes contact at least one body region of the person doing training. The voltage supply to the electrodes is activated during the training by means of a control unit that may be affixed to the article of clothing. For attaching the control unit to the article of clothing, the article of clothing is provided with a carrier unit that is electrically connected to the electrodes. The control unit may be latched and fastened to this carrier unit by means of fixing elements.

### SUMMARY OF THE INVENTION

It is therefore an object of the invention to provide an improved device for electrical muscle stimulation training, using an article of clothing for stimulating body regions of a user. In addition, a method for the electrical stimulation of body regions is provided.

In an example, the device for stimulating body regions of a user comprises an article of clothing made of an elastic textile structure with integrated electrodes, as well as a local control unit for controlling and/or supplying power to the electrodes.

Integrated into this control unit, in addition to further components, is a voltage source, for example a lithium-ion rechargeable battery, for supplying power to the electrodes. For trouble-free operation of the device, during the training a continuous supply of current or voltage as well as control of the electrodes of the article of clothing must be ensured. The control unit must therefore be securely and fixedly connected to the article of clothing so that stable contact between the electrodes of the article of clothing and the control unit is provided, even in the event of intense, jerky movements by the user.

In particular, according to the invention the article of clothing may have a pocket that is designed in particular as a closable receiver, in which the control unit is accommodated when the article of clothing is being used. Situated in an inner area of the receiver or the pocket is a contacting unit to which the electrodes are electrically connected via supply lines, and to which the control unit may be electrically coupled. Since the control unit is accommodated in a pocket or a closable receiver of the article of clothing, it is ensured that a secure connection of the voltage supply of the control unit to the supply lines to the electrodes is maintained, even for activities with intense movements such as jumping on a trampoline, jogging, or complex yoga or HIIT exercises.

The receiver or the pocket is advantageously situated in the chest or hip region of the article of clothing, i.e., in an area where it does not impair the user's freedom of movement.

The control unit can be fixedly, i.e., nondetachably, connected to the contacting unit. The receiver or the pocket may advantageously be designed as a cavity in the article of clothing that is closed on all sides, so that the control unit is nondetachably integrated into the article of clothing and inaccessible from the outside. The control unit is advantageously encapsulated, for example provided with a plastic skin, to protect it from penetration of perspiration, water, etc., and to allow cleaning of the article of clothing, for example washing it in a washing machine, without damaging the control unit. In this case, charging of the voltage source integrated into the control unit advantageously takes place inductively before or after the article of clothing is used, for example using an inductive charging station on which the article of clothing is laid or stretched.

As a further example to an example that is closed on all sides, the receiver may also be a pocket with an (advantageously closable) opening that is woven into or sewn onto the article of clothing, or affixed in a similar manner. The opening allows accessibility to the control unit from the outside, so that individual components of the control unit, in particular a rechargeable battery that serves as a voltage source and is inserted into the control unit, may be removed. In this way, the rechargeable battery may be charged outside the article of clothing, inductively or by insertion into a charging unit, before or after the article of clothing is used.

The control unit can be detachably connected to the contacting unit or to a plug connection. The pocket of the article of clothing has an opening through which the control unit may be removed from the article of clothing, for example to charge or replace the voltage source that is integrated into the control unit, to use the control unit in another article of clothing, to perform maintenance on the control unit, etc. To secure the position of the control unit in the pocket of the article of clothing during training, the pocket can be advantageously provided with a closure system, for example a zipper, a hook and loop fastener, a push button, etc.

The electrical connection of the control unit to the contacting unit in the interior of the pocket advantageously can take place by means of a plug connection and/or by magnetic positioning of the corresponding contact elements on the control unit and the contacting unit.

The receiver may also be designed as a watertight, closable container that is fixedly connected to the article of clothing, and into which the control unit is or may be inserted. The container may in particular be made up of two plastic shells that are connected by hinges, a circumferential seal being provided between the plastic shells to prevent penetration of water, perspiration, etc., into the interior of the container in the closed state of the container. The contacting unit, which may be fixedly or detachably connected to the control unit, is situated in the interior of the container.

In addition to the voltage source, a module for data transfer can be integrated into the control unit, via which the control unit may be activated from a base station, for example a mobile telephone. The data transfer may advantageously take place on the one hand via Bluetooth (for household applications), or on the other hand via Zigbee (for studio applications) and/or via some other standard, wherein the control unit itself recognizes and appropriately selects the data transfer mode that is suitable for the particular environment. As an alternative to the integration into the control unit, the data transfer module may be integrated directly into the article of clothing.

The device may also include a visual and/or acoustic stimulation unit that is configured to provide a user with visual or acoustic stimuli based on signals that are transmitted from the base station. As mentioned above, the base station may in particular be a mobile terminal such as a mobile telephone, or a tablet in conjunction with an appropriate app.

The base station may be configured to transmit signals and frequencies for generation of stimuli, which influence the intensity of EEG frequency bands, i.e., in the delta, theta, beta, or gamma frequency range, for example. In this way, by using an application on a mobile terminal, in addition to the electrodes on an article of clothing, headphones and/or light sources, for example LEDs in eyeglasses, may be rhythmically activated in such a way that the appropriate desired frequency pattern occurs in an EEG.

When the base station is configured to transmit signals for generating acoustic and/or optical stimuli which are coordinated with the activation of the electrodes, in conjunction with the simultaneously activated electrodes of the article of clothing the training motivation and effectiveness may be further increased.

The described variant is suited in particular for bringing the brain into a certain state in order to convey learning content by suggestion, or to achieve something similar.

In other words, the device may be configured to simultaneously generate periodic stimulations of a user in several different ways, in particular via acoustic stimulation, for example by means of headphones, optical stimulation, for example by means of eyeglasses equipped with light sources, in particular LEDs, electrical stimulation, for example via the electrodes of an EMS suit, tactile stimulation, for example via an appropriate actuator system which may be integrated into an EMS suit, for example, and/or magnetic stimulation, for example via in particular periodic magnetic fields generated by a magnetic field mat

As a result of coordinating the frequencies of the particular stimulations with one another, in particular with the frequencies being similar or identical or integer multiples of one another and adapted to one another with regard to their phase position so that a form of common mode results, increased effectiveness may be achieved when generating a desired state. For example, the user's brain may be shifted into a particular frequency range via which increased potential for “superlearning” or other methods of cognitive conditioning may be achieved.

It is self-evident that in addition to a combination of all the options stated above, subcombinations of the options may also be meaningfully applied.

If the article of clothing is part of the features at a fitness studio in which multiple persons simultaneously train, an identification module, for example an NFC chip, may additionally be affixed in the chest, shoulder, or hip region of the article of clothing. This identification module is used to allow the article of clothing (or its user at the time) to be assigned to a selected training program in a base station of the studio.

Furthermore, sensors may be integrated into the article of clothing, via which training-related measured values of the physical condition of the user (for example, blood pressure, pulse rate, lactate value, etc.) are detected. The measured values from the sensors are advantageously evaluated online in the control unit and/or transmitted to the base station via the data transfer module integrated into the control unit. This allows training-related monitoring of the user's medical data and simultaneous intervention, for example in the case of overexertion by the user, as well as checking for whether certain training or treatment procedures are being correctly performed. In addition, the training may be optimized for the user's physical condition, for example by operating selected electrodes in a mode that results in an optimal training outcome in the shortest time possible or with the least possible physical effort.

Alternatively or additionally, actuators may be integrated into the article of clothing, via which tactile stimuli (for example, touch, pressure, temperature, etc.) may be transferred to the skin of the user. The article of clothing may thus be used as part of a virtual environment in which a user may take part in different types of sports in a virtual setting, train in a virtual work environment, or receive therapy under realistic conditions.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a schematic front view of an article of clothing for electrical muscle stimulation (EMS) training of selected body regions of a user;

FIG. 2a shows a schematic detailed view of a control unit that is accommodated in a pocket of the article of clothing according to area II-II in FIG. 1;

FIG. 2b shows a schematic sectional view of the pocket from FIG. 2a according to the section line IIb-IIb in FIG. 2a;

FIG. 3 shows a schematic sectional view of an example of the pocket; and

FIG. 4 shows a schematic detailed view of a control unit that is accommodated in a container that is connected to the article of clothing.

### DETAILED DESCRIPTION

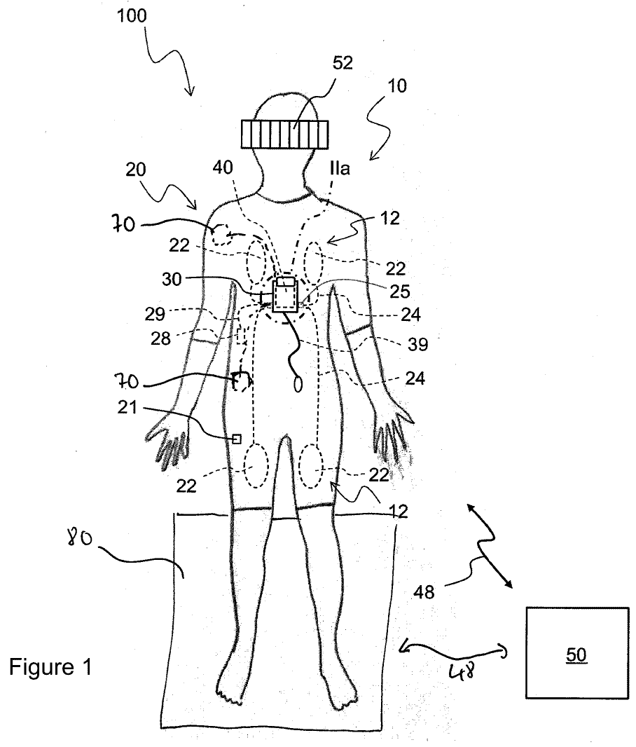

FIG. 1 shows a schematic illustration of a user 10 when using a device 100 according to the invention for electrical muscle stimulation of selected body regions, for which purpose the user 10 wears an article of clothing 20 in the form of a bodysuit with short sleeves and short legs. The article of clothing 20, at least in sections, is made of an elastic textile material, and on an inner side facing the user 10 contains several pairs of electrodes 22 that are situated in selected body regions 12 of the user 10 and whose outer contours are indicated by dashed lines in FIG. 1. The pair of electrodes 22 is situated at the inner side of the article of clothing 20 in such a way that during use, the electrodes contact the skin of the user 10 in the body regions 12 to be stimulated. The pair of electrodes 22 may be formed, for example, by flexible conductive films that are fastened to the inner side of the article of clothing 20. Alternatively, the electrodes may be formed by a conductive elastic yarn that is locally incorporated, for example knitted or embroidered, into the (electrically nonconductive) elastic material of the article of clothing 20. In the example in FIG. 1, the pair of electrodes 22 is integrated in the region of the right and left thighs of the user 10, and a pair of electrodes 22 is integrated in the chest region of the user 10. Further sensors 28 are incorporated into the article of clothing 20, via which training-relevant measured variables or movement data of the user are determined, for example BIA sensors, moisture sensors, blood sugar sensors, lactate sensors, contact sensors, movement sensors, magnetoresistive sensors, etc. One of these sensors 28 is schematically illustrated by dashed lines in FIG. 1. For contacting the electrodes 22 and the sensors 28, electrical supply lines 24, 29 are provided which are integrated into the textile of the article of clothing 20, and which are connected to a contacting unit 25, for example a flat ribbon cable, that is likewise integrated into the textile of the article of clothing.

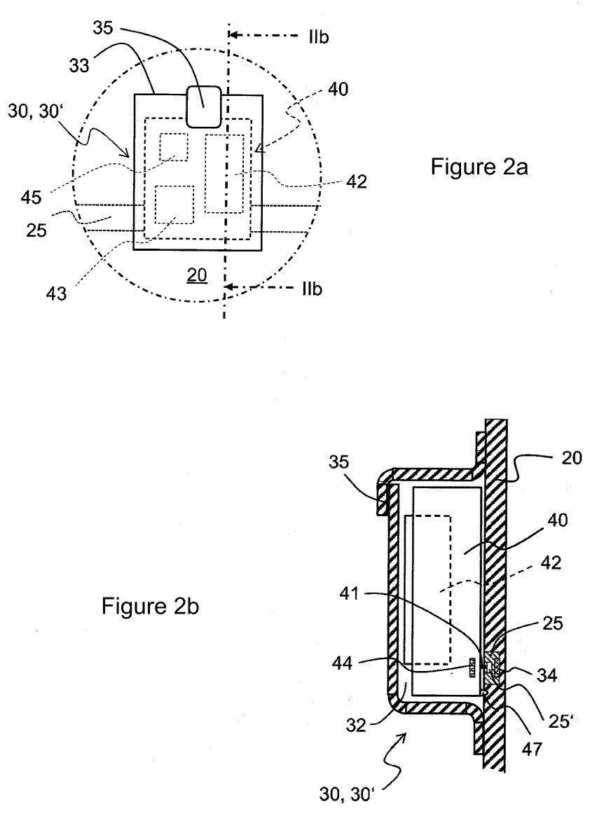

For activating and supplying voltage to the electrodes 22 and for supplying voltage and detecting measured values for the sensors 28, a control unit 40 is provided which contains a voltage source in the form of a rechargeable battery 42, for example a lithium-ion rechargeable battery, via which the electrodes 22 and sensors 28 are supplied with power (see the detailed view in FIG. 2a). In addition, the control unit 40 contains a microcontroller 43 via which predefined excitation cycles of the electrodes 22 may be controlled, and measured values from the sensors 28 may be detected and stored. Furthermore, the control unit 40 contains a module 45, for example a Bluetooth module or a Zigbee module, for exchanging data with a base station 50. For example, a certain training program may thus be transferred to the control unit 40 without contact, or user data that are obtained during the training via the sensors 28 that are integrated into the article of clothing 20 may be transferred to the base station 50 without contact. Such a data transfer or data exchange is symbolically denoted by a double arrow 48 in FIG. 1.

During use of the article of clothing 20, the control unit 40 must be continuously electrically connected to the electrodes 22 in order to ensure the desired activation and voltage supply for the electrodes 22. For this purpose, the article of clothing 20 is provided with (at least) one closable receiver 30 in which the control unit 40 is accommodated, and in the interior 32 of which the control unit 40 is connected to the contacting unit 25. This connection may be established in particular by a plug connection or a detent connection.

Furthermore, FIG. 1 shows a magnetic field mat 80 by means of which electromagnetic stimulation for the user 10 may be generated. The data exchange with the magnetic field mat may take place wirelessly via the control unit 40; alternatively or additionally, it is likewise conceivable to activate the magnetic mat 80 directly from the base station 50, as indicated by the arrow 48.

In an example shown in FIGS. 2a and 2b, the receiver 30 is a pocket 30′ that is incorporated into or placed on the textile of the article of clothing 20. The control unit 40 is provided with a plurality of pin-shaped electrical contact elements 41 that protrude from the control unit 40 and come into contact with the contacting unit 25, situated in the inner area 31 of the pocket 30′, when the control unit 40 is inserted into the pocket 30′. To establish an electrical connection, the contacting unit 25 is incorporated into the article of clothing 20 in such a way that contact fields 25′ of the contacting unit 25, into which the supply lines 24, 29 open (and which are thus in electrical connection with the electrodes 22 and the sensors 28), are opposite the contact elements 41 of the control unit 40 in the inner area of the pocket 30′, so that the contact elements 41 of the control unit 40 may be connected to these contact fields 25′ of the contacting unit 25. It is important to ensure the correct pairing of associated contact elements 41 and contact fields 25′. The connection may be established, for example, by means of a flat ribbon cable plug connection or some other plug system; in particular, the control unit 40 may be inserted into a receiving shoe that is provided in the pocket 30′, and which via mechanical fixing of the control unit 40 ensures a correct connection of the contact elements 41 of the control unit 40 to the contact fields 25′ of the contacting unit 25. Alternatively, as shown in FIG. 2b, the contacting unit 25 as well as the control unit 40 may be provided with magnets 34, 44 that ensure correct positioning of the individual contact elements 41 of the control unit 40 with respect to the associated contact fields 25′ of the contacting unit 25. In addition, for example locking tabs 47 which allow mechanical positioning of the control unit 40 with respect to the contacting unit 25 may be provided.

To captively accommodate the control unit 40 in the pocket 30′, the pocket 30′ is advantageously provided with a closure system 35, for example a zipper or a hook and loop strip. Such a closure system 35 holds the control unit 40 in the desired position during use of the article of clothing 20, but at the same time ensures that the control unit 40 may be quickly and easily removed from the pocket 30′ of the article of clothing 20, so that on the one hand the battery 42 that is integrated into the control unit 40 may be charged before/after the training, and on the other hand the article of clothing 20 may be cleaned or washed without damaging the control unit 40.

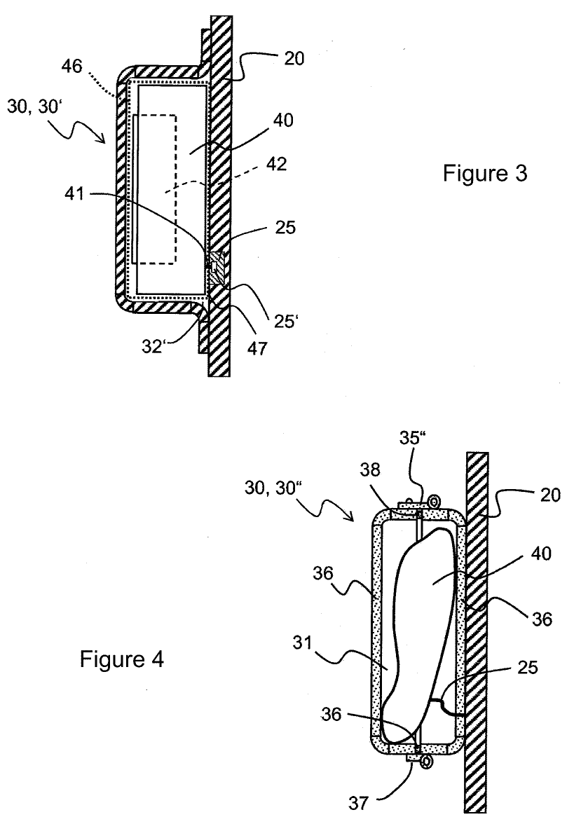

In an example illustrated in FIG. 3, the control unit 40 is fixedly, i.e., nondetachably, integrated into the article of clothing 20, so that the control unit 40 together with the rechargeable battery 42 is an integral part of the article of clothing 20. The pocket 30′ then forms a closed cavity 32 in which the control unit 40 is accommodated. The control unit 40 is enclosed by a watertight case 46 to protect the control unit 40 (including the rechargeable battery 42) from damage due to water, perspiration, etc. This protective case 46 is designed in such a way that the operability of the control unit 40 is not impaired during regular cleaning of the article of clothing (for example, in a washing machine at 30° C.).

Since the rechargeable battery 42 in this example cannot be removed from the article of clothing 20, the charging of the rechargeable battery 42 takes place inductively.

In a further example, the control unit 40 can be fixedly, i.e., nondetachably, integrated into the article of clothing 20, but the rechargeable battery or battery 42 installed in the control unit 40 is detachably connected to the control unit 40. The pocket 30′ has an opening, so that the rechargeable battery/battery 42 may be removed from the pocket 30 for charging or replacement.

FIG. 4 shows a receiver 30 in the form of a watertight, closable container 30″ that is fixedly connected to the article of clothing 20. The container 30″ comprises two half shells 36, made of plastic, that are joined together by a hinge 37, and in the closed state of the container 30″ are fixed relative to one another by means of a closure 35″. A circumferential sealing ring 38 that prevents penetration of water, perspiration, etc. into the interior 31 of the container 30″ is provided between the half shells 36. The contacting unit 25 is connected via a flat ribbon cable plug connection to the control unit 40 accommodated in the container 30″. As illustrated in FIG. 4, the control unit 40 does not have to have the rectangular shape shown in FIGS. 2 and 3, and instead may have any desired (prismatic or free-form) shape; in particular, the control unit may have the form of a three-dimensional figure or a three-dimensional logo, may be customized for the user, may be provided with a soft or textile surface, etc. The control unit 40 may also have function lights that advantageously depict a logo and which light up green, for example. For this purpose, the wall of the container 30″ (or the material in the region of the pocket 30′) may be transparent or designed as a mesh insert.

The receiver 30 for the control unit 40 may in particular be situated centrally in the chest region, or at the side in the hip region of the article of clothing 20. An emergency stop switch in the form of a pull cord 39 is provided in the immediate vicinity of the receiver 30, and may be employed by the user 10 to interrupt the training program.

The control unit 40 has function lights that may advantageously depict a logo and light up green, for example. For this purpose, the material in the region of the pocket 30 may be transparent or designed as a mesh insert.

An identification module 21, for example an NFC chip, may be affixed in the article of clothing 20. This identification module 21 is used to allow the article of clothing 20 (or its user) to be assigned to a selected training program in a base station 50 of the studio. By use of this identification module 21, a specific training program that is customized for the particular user may be sent via the base station 50 to the control unit 40 of the article of clothing 20 and executed there.

To increase the wearing comfort, the article of clothing 20 may be provided with mesh inserts under the arms and in the crotch.

By means of the sensors 28 that are integrated into the textile of the article of clothing 20, process-related user data (for example, pulse rate, blood pressure, blood sugar level, oxygen saturation in the blood, local body temperature, etc.) may be detected during the training. For muscle tenseness and/or increased muscular activity, these sensors 28 may measure the activity and transfer the information to the base station 50, where these measured data are evaluated or compared to target data and possible countermeasures are initiated. This allows an optimized training, for example for a marathon runner who would like detect his/her continuous stress and optimally adjust it.

By use of the measured values from suitable integrated sensors 28, it is possible, for example by biofeedback or other appropriate techniques, to determine the percentage and type of activation of the individual muscles of the user 20 during the training, and which muscle groups are activated and to what extent, so that the stimulation may be set even more precisely or may also be automated. For example, a report that “95% of the white skeletal musculature in the quadriceps is stimulated” may be interpreted and evaluated differently, and the associated electrode excitation may be correspondingly adapted.

In addition, by use of further integrated sensors 28 the position and/or movements and/or acceleration of individual components of the article of clothing may be detected. This provides the option, possibly in conjunction with a virtual environment or augmented reality, to transmit appropriate information concerning the movement of the user and its intensity to the base station 50, so that a realistic movement may be simulated in practically any given real or virtual environment. The user 20 may thus interact with a virtual environment, for example a virtual trainer, for example with the aid of 3D glasses 52 (indicated in FIG. 1). The glasses 52 may also include headphones or earbuds for generating acoustic signals. Measured values that provide information about the correct performance of the movement to be executed may be detected by means of the sensors 28. With the aid of such feedback, a wide variety of movement sequences may be learned and optimized, for example in training for athletic activities, for learning and optimizing work processes, in game situations, etc. Alternatively or additionally, actuators 70 may be integrated into the article of clothing 20, via which tactile stimuli (for example, touch, pressure, temperature, etc.) may be transferred to the skin of the user 10 in order to simulate a training environment that is even closer to reality. These actuators 70 are schematically indicated in FIG. 2 strictly by way of example. In such a virtual environment, there may be a desire to simulate special stresses on individual muscles via electrical frequencies that are transmitted via the electrodes in the article of clothing. Thus, for example, virtual skiing on a mogul slope, jogging, cycling (also downhill), or a virtual fitness studio with virtual weights and machines may likewise be simulated, similarly as for riding on a roller coaster.

The control unit 40 may also be equipped with a WLAN chip that allows a remote person, for example a trainer, or remote hardware/software to control the intensity of the electrical stimulation of individual muscles from a distance, and/or to read out data concerning the physical condition of the user 10 (for example, pulse rate, blood pressure, etc.). In this way, a user 10 of the article of clothing 20 may take part in virtual training, competitions, or games and thereby experience the most realistic perception, control, stress, and movement possible.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A device for stimulating body regions of a user, the device comprising:
an article of clothing made of an elastic textile structure with integrated electrodes and integrated electrical supply lines connectable to the electrodes;
a local control unit; and
a contacting unit that is electrically connected to the supply lines and to the electrodes.

2. The device according to claim 1, wherein the article of clothing has a closable receiver for accommodating the control unit.

3. The device according to claim 1, wherein the receiver is a pocket that is integrated into the article of clothing.

4. The device according to claim 2, wherein the pocket is provided with a closure system in the form of a zipper.

5. The device according to claim 1, wherein the receiver is a watertight, closable container that is connected to the article of clothing.

6. The device according to claim 1, wherein the control unit is fixedly connected to the contacting unit.

7. The device according to claim 1, wherein the control unit is detachably connected to the contacting unit.

8. The device according to claim 1, wherein the control unit includes a module for data transfer between the control unit and a base station.

9. The device according to claim 1, wherein an identification module is integrated into the article of clothing.

10. The device according to claim 1, wherein at least one sensor is integrated into the article of clothing.

11. The device according to claim 1, wherein at least one actuator is integrated into the article of clothing via which a tactile stimulus is applied to the skin of the user.

12. The device according to claim 1, wherein the device is configured to simultaneously generate periodic stimulations of a user in several different ways.

13. The device according to claim 12, wherein the device is configured to generate periodic stimulations of a user by acoustic stimulation.

14. The device according to claim 12, wherein the device is configured to generate periodic stimulations of a user by optical stimulation.

15. The device according to claim 12, wherein the device is configured to generate periodic stimulations of a user by electrical stimulation.

16. The device according to claim 1, wherein the device is configured to generate periodic stimulations of a user by magnetic stimulation.

17. The device according to claim 12, wherein the device is configured to generate periodic stimulations of a user by tactile stimulation.

18. A method for stimulating body regions of a user, the method comprising:
providing the user with an article of clothing made of an elastic textile structure with integrated electrodes and integrated sensors; and
connecting integrated electrodes and integrated sensors to a control unit.

19. The method according to claim 18, wherein measured values of the user are detected by integrated the sensors and evaluated with the aid of the control unit, and wherein an excitation of the electrodes takes place using these measured values.

20. The method according to claim 18, wherein periodic stimulations of a user are generated, wherein the periodic stimulations of the user are generated by acoustic stimulation, wherein the periodic stimulations of the user are generated by optical stimulation, wherein the periodic stimulations of the user are generated by electrical stimulation, wherein periodic stimulations of the user are generated by magnetic stimulation, and/or wherein periodic stimulations of the user are generated by tactile stimulation.
